(19) United States
(12) Patent Application Publication
(10) Pub. No.: US 2022/0002667 A1
(43) Pub. Date: Jan. 6, 2022

(54) BACILLUS SP. PROBIOTIC STRAINS AND MUTATIONS

(71) Applicant: DSM IP ASSETS B.V., HEERLEN (NL)

(72) Inventors: Lisa LAPRADE, Kaiseraugst (CH); Ghislain SCHYNS, Kaiseraugst (CH)

(21) Appl. No.: 17/292,683

(22) PCT Filed: Nov. 8, 2019
(86) PCT No.: PCT/EP2019/080630

§ 371 Date: May 10, 2021

Related U.S. Application Data
(60) Provisional application No. 62/758,821, filed on Nov. 12, 2018.

(30) Foreign Application Priority Data
Nov. 14, 2018 (EP) 18206180.4

(51) Int. Cl. C12N 1/20; A23K 10/16

(52) U.S. Cl. CPC C12N 1/205; C12R 2001/125; A23K 10/16

(57) ABSTRACT

The invention involves mutant genes that enable a Bacillus or other bacterial strain, such as Bacillus subtilis or B. licheniformis strain to overproduce acids. These genes can be inserted into a suitable strain and the resulting mutant strain can be used as a probiotic, preferably for use in animal health.

### FIELD OF THE INVENTION

The invention involves mutant genes that enable a Bacillus or other bacterial strain, such as Bacillus subtilis or B. licheniformis strain to overproduce acids. These genes can be reproduced via mutagenesis, or inserted into a suitable strain and the resulting mutant strain can be used as a probiotic, preferably for use in animal health.

### BACKGROUND OF THE INVENTION

The use of B. subtilis strains as probiotic ingredient in the feed industry is well known in the art. The function of probiotics (also called “direct-fed microbials” or “DFM”) is to influence the gut microflora in a positive way by supporting the growth of beneficial bacteria and/or the suppression of the growth of pathogenic bacteria. Ideally, by using probiotics, the use of antibiotic growth promotors (AGPs) becomes redundant. It is also desirable for probiotics to fulfill other functions, e.g., helping in the digestion of specific feed ingredients. Thus, there is a need for probiotics which influence the gut microflora in a positive way and fulfill other functions.

### DETAILED DESCRIPTION OF THE INVENTION

It has been found, in accordance with this invention, that certain mutations which can be induced in a B. subtilis bacteria are beneficial in that various acids can be overproduced, such as acetate. Furthermore, these mutations have been characterized and the mutant portions of the various genes can be inserted into new bacteria, or the mutations can be induced in another Bacillus, such as B. subtilis or B. licheniformis, to improve an existing strain, or to create a new probiotic strain with this beneficial property.

One embodiment of the present invention is directed to a new strain of Bacillus sp. bacteria which is effective at inhibiting the growth of Clostridium perfringens (one of the major commercially relevant pathogens of poultry), preferably a B. subtilis or a B. licheniformis. It possesses beneficial mutations promoting acetate overproduction.

In another embodiment, the current invention is directed to a Bacillus subtilis strain and preparations that contain or are derived from this strain. Specifically, the invention is directed to one or more of the following groups:

The Bacillus sp., preferably a B. subtilis or B. licheniformis strain, of this invention exhibits at least one of the following characterizing sequences:

a) a 16S rDNA sequence with a sequence identity of at least 95%, and preferably, 100%, to the polynucleotide sequence according to SEQ ID NO:15, designated BMS7 16S;

b) an alsS sequence with a sequence identity of at least 95%, and preferably, 100%, to the polynucleotide sequence according to SEQ ID NO:2 designated alsS (5.1) or SEQ ID NO:3 designated alsS (5.2);

c) an adhA sequence with a sequence identity of at least 95%, and preferably, 100%, to the polynucleotide sequence according to SEQ ID NO:9 designated adhA (5.1);

d) an bdhA sequence with a sequence identity of at least 95%, and preferably, 100%, to the polynucleotide sequence according to SEQ ID NO:7 designated bdhA (5.1);

e) a dhbA sequence with a sequence identity of at least 95%, and preferably, 100%, to the polynucleotide sequence according to SEQ ID NO:13 designated dhbA (1.6).

f) a pta sequence with a sequence identity of at least 95%, and preferably, 100%, to the polynucleotide sequence according to SEQ ID NO:5 designated pta (5.1).

A further embodiment is A Bacillus sp. strain which comprises at least one of the following characterizing sequences:

a) a 16S rDNA sequence with a sequence identity of at least 95%, and preferably, 100%, to the polynucleotide sequence according to SEQ ID NO:15, designated BMS7 16S;

b) a adhA sequence with a sequence identity of at least 95%, and preferably, 100%, to the polynucleotide sequence according to SEQ ID NO:9 designated adhA (5.1);

c) an bdhA sequence with a sequence identity of at least 95%, and preferably, 100%, to the polynucleotide sequence according to SEQ ID NO:7 designated bdhA (5.1);

d) a dhbA sequence with a sequence identity of at least 95%, and preferably, 100%, to the polynucleotide sequence according to SEQ ID NO:13 designated dhbA (1.6).

e) a pta sequence with a sequence identity of at least 95%, and preferably, 100%, to the polynucleotide sequence according to SEQ ID NO:5 designated pta (5.1).

Another embodiment is A Bacillus strain comprising an alsS sequence with a sequence identity of at least 95%, and preferably, 100%, to the polynucleotide sequence according to SEQ ID NO:2 designated alsS (5.1) or SEQ ID NO:3 designated alsS (5.2) and at least a second sequence selected from the group consisting of:

a) a 16S rDNA sequence with a sequence identity of at least 95%, and preferably, 100%, to the polynucleotide sequence according to SEQ ID NO:15, designated BMS7 16S;

b) a alsS sequence with a sequence identity of at Least 95%, and preferably, 100%, to the polynucleotide sequence according to SEQ ID NO:2 designated alsS (5.1) or SEQ ID NO:3 designated alsS (5.2);

c) a adhA sequence with a sequence identity of at least 95%, and preferably, 100%, to the polynucleotide sequence according to SEQ ID NO:9 designated adhA (5.1);

d) an bdhA sequence with a sequence identity of at least 95%, and preferably, 100%, to the polynucleotide sequence according to SEQ ID NO:7 designated bdhA (5.1);

e) a dhbA sequence with a sequence identity of at least 95%, and preferably, 100%, to the polynucleotide sequence according to SEQ ID NO:13 designated dhbA (1.6).

f) a pta sequence with a sequence identity of at least 95%, and preferably, 100%, to the polynucleotide sequence according to SEQ ID NO:5 designated pta (5.1). Yet another embodiment is a Bacillus strain comprising an alsS sequence with a sequence identity of at least 95%, and preferably, 100%, to the polynucleotide sequence according to SEQ ID NO:2 designated alsS (5.1) or SEQ ID NO:3 designated alsS (5.2) and further comprising all the sequences a-f.

The aforementioned sequences can be inserted into a desired Bacillus strain, or induced in a Bacillus strain, preferably a Bacillus subtilis or B. lichenformis, and more preferably a B. subtilis to create novel strains which have beneficial probiotic activities. In one particularly preferred embodiment, all the sequences above which have a sequence identity of at least 95%, and preferably 100% of SEQUENCE ID NOS 15, 9, 7, 13, 5 and one of either SEQ ID NO 2 or 3 are present in the same bacteria. One such example of this bacteria is designated BMS 7.

In one embodiment of this invention. The Bacillus sp. strains are further characterized by being able to overproduce acetic acid or acetate. In one embodiment, the strain is a B. subtilis strain wherein said strain is capable of overproducing acetic acid or acetate by at least 40%, 50%, 60%, 70%, 80%, 90%, 100% compared to the wild type strain BMSS. One preferred embodiment is BMS7.

Another embodiment of this invention is a composition comprising the Bacillus strains of this invention or compounds obtained from the Bacillus of this invention. The composition may be a feed-stuff and further comprises at least one further feed or food ingredient selected from the group consisting of: proteins, carbohydrates, fats, further probiotics, prebiotics, enzymes, vitamins, immune modulators, milk replacers, minerals, amino acids, coccidiostats, acid-based products, medicines, and combinations thereof.

Without wishing to be bound by any theory, it is thought that the Bacillus sp. strains according to the current invention enhance animal health by a multifaceted mode of action, including the production of antibacterial metabolites with selective efficacy and the competition with pathogenic bacteria by better consuming the available nutrients, thereby suppressing effective establishment of pathogenic bacteria in the gut.

Probiotics are considered more advantageous than antibiotics because they do not destroy bacteria indiscriminately and do not they lead to antibiotic resistant strains of pathogenic bacteria. Normally, the probiotic bacteria selectively compete with pathogenic bacteria by production of antimicrobial substances with specific efficacy and are ideally able to simultaneously enhance the growth and viability of beneficial gut microflora. Further, they are preferably able to stimulate a systemic immune response in the treated animals.

The mutant strains of the current invention are preferably mutants of existing strains. The term “mutant strain” used throughout refers to mutants that arise from a wild type Bacillus, such as BMS5 whose genome is given in SEQ ID NO:1, with the intentional use of mutagens such as those known in the art, and preferably EMS. Such mutants may be obtained by classical methods, such as growing the Bacillus subtilis strain in the presence of UV light or in the presence of a certain antibiotic to which the parent is susceptible and testing any resistant mutants for improved biological activity or improved ability to enhance one or more of the indicia of animal health. Other methods for identifying mutants are known to those of ordinary skill in the art. But besides these preferred mutants all other kinds of mutants e.g., mutants obtained by genetic engineering, are also part of the current invention.

Another embodiment of the current invention is a Bacillus sp. mutant of the strain BMS5 which is not found in nature and has the characteristics mentioned above.

In a preferred embodiment of the current invention, the strains and preparations of the present invention are administered orally to animals.

A further subject of the current invention is also the use of a B. subtilis strain and/or a Bacillus licheniformis in the preparation of the current invention as a probiotic ingredient (DFM) in feed products. The Bacillus subtilis or B. licheniformis strains of the current invention and compositions containing them, when administered to animals, preferably enhance the health of such animals and/or improve the general physical condition of such animals and/or improve the feed conversion rate of such animals and/or decrease the mortality rate of such animals and/or increase the survival rates of such animals and/or improve the weight gain of such animals and/or increase the productivity of such animals and/or increase the disease resistance of such animals and/or increase the immune response of such animals and/or establish or maintain a healthy gut microflora in such animals and/or reduce the pathogen shedding through the feces of such animals In particular the strains and compositions of the current invention might be used to assist in re-establishing a healthy balance of the gut microflora after administration of antibiotics for therapeutic purposes. In some preferred embodiments, the animals are poultry.

A further subject of the current invention is therefore a method of enhancing the health of animals and/or of improving the general physical condition of animals and/or of improving the feed conversion rate of animals and/or of decreasing the mortality rate of animals and/or of increasing the survival rates of animals and/or of improving the weight gain of animals and/or of increasing the productivity of animals and/or of increasing the disease resistance of animals and/or of increasing the immune response of animals and/or of establishing or maintaining a healthy gut microflora in animals and/or of reducing the pathogen shedding through the feces of animals, wherein the strains and/or preparations of the current invention or the compositions of the current invention, which comprise such strain(s), are administered to animals. In some preferred embodiments, the animals are poultry.

A further subject of the current invention is therefore also the use of strains and/or preparations and/or compositions of the current invention for enhancing the health of animals and/or for improving the general physical condition of animals and/or for improving the feed conversion rate of animals and/or for decreasing the mortality rate of animals and/or for increasing the survival rates of animals and/or for improving the weight gain of animals and/or for increasing the productivity of animals and/or for increasing the disease resistance of animals and/or for increasing the immune response of animals and/or for establishing or maintaining a healthy gut microflora in animals and/or for reducing the pathogen shedding through the feces of animals, wherein the strains and/or preparations of the current invention or the compositions of the current invention, which comprise such strain(s), are administered to animals. In some preferred embodiments, the animals are poultry.

A further subject of the current invention is therefore also the strains and preparations of the current invention as mentioned before and the compositions of the current invention, containing those strains, for enhancing the health of animals and/or for improving the general physical condition of animals and/or for improving the feed conversion rate of animals and/or for decreasing the mortality rate of animals and/or for increasing the survival rate of animals and/or for improving the weight gain of animals and/or for increasing the productivity of animals and/or for increasing the disease resistance of animals and/or for increasing the immune response of animals and/or for establishing or maintaining a healthy gut microflora in animals and/or for reducing the pathogen shedding through the feces of animals. In some preferred embodiments, the animals are poultry.

The general procedure for producing bacterial strains of this invention is summarized as follows. Bacterial strains have been mutagenized using ethyl methyl sulfonate (EMS). Different concentrations and times of exposure to the chemical have been tested to ensure a ratio of mutations around 20/cell. To hit successfully a biochemical pathway (like the one for organic acids production that we are targeting), it is important to have more than one mutation, but not too many since this become detrimental to the viability of cells.

After exposure to the mutagen, cell viability (so called killing rate) and auxotrophy for amino acids are tested. Too many mutations are detrimental for such physiological processes as sporulation (probiotics for ANH are sporeformers and sold as spores), prototrophy (important for fermentation), and maybe probiotic behavior in the gut. All these techniques, their parameters and concerns are known from people skilled in the art.

Banks of mutagenized Bacillus subtilis BMS5 bacterial cells were then screened on rich medium plates using a pH indicator (bromophenol blue) since the phenotype we were looking for induces acids production/excretion. The indicator we used makes cells producing acids turning yellow. A large number of phenotypically yellow colonies were collected based on the intensity of the yellow color and the timing of appearance of the yellow color. Positively yellow colonies were restreaked for isolation on identical medium. Yellow mutants at that point were then cultured in liquid medium to be tested by HPLC for the organic acids they were producing. Lactic, butyric, acetic and propionic acids production were evaluated for around 30 colonies on liquid chromotography. Two colonies were identified as strong acetic acid producers. They were named BMS5-1 and BMS-2. Both isolates were then evaluated for their ability to sporulate, which is a critical parameter for process (fermentation). One of the strain (BMS5-2) was observed not to sporulate as efficiently as the other. Its use was discontinued since it would need to be produced at much higher volume to yield the same number of spores.

BMS5-1 (now designated as BMS7) was then cultured to extract chromosomal DNA for genome sequencing. Genome sequencing was performed using Illumina technology.

Genome mutations were then identified between 13MS7 and its parental strain BMS5. The genes harboring mutations linked to acetate production pathway are listed in the examples.

Resistance to antibiotics of BMS7 and BMS5 (parental strain) were then assessed to be sure that no mutation was increasing resistance to the 10 antibiotics listed by EFSA for Bacillus strains.

The following non-limiting Examples are presented to further illustrate the invention.

### EXAMPLES

Bacterial growth conditions

Bacillus strains were grown on minimal medium (MM; 1 X Spizizen salts, 0.04% sodium glutamate, and 0.5% glucose) or veal infusion-yeast extract complete medium (VY) or grown on agar plates consisting of tryptose blood agar base (TBAB, Difco, Md.).

Growth was performed at 37° C.

Isolation of EMS-induced acids-producing mutants

Banks of ethyl methylsulfonate (EMS; d=1.21 g/ml solution) were prepared and screened. Logarithmic-stage BMS5 cells were treated with 460 mM EMS for 60 min, and aliquots were frozen in 10% glycerol at −80° C. The killing rate was determined by plating serial dilutions of cells treated with EMS vs cells with no treatment but the same procedure on TBAB plates and comparing CFUs. Cells from the frozen stock were diluted 1:5 in VY medium, incubated at 37° C. for 60 min and plated onto TBAB medium containing 2% glucose and bromocresol purple as pH indicator. This pH indicator is purple above pH 6.8 and yellow below pH 5.2 (pKₐ=6.3). Phenotypically diverse yellow colonies were collected along 24 hours at 37° C. Yellow colonies were re-streaked for isolation on similar medium and cultured in liquid medium VY with and without glucose before characterization of organic acid profile on HPLC. Prior to organic acid screening, spore forming ability was assessed by 30 min exposure to 90° C. prior to plating on TBAB in comparison to non-heated cells.

Characterization of organic acids profile Lactic, propionic, butyric and acetic acids were quantified. Method for acid quantitation uses and HPLC (high pressure liquid chromatograph) system with UV (ultra-violet) and RI (refractive index) detection. Majority of quantitation is conducted using RI, unless there is a co-elution detected, where UV at 210nm will be used. Column is a BioRad Aminex HPX-87H 300×7.8mm with HPX-87H guard column. Solvent is 0.05% Trifluoroacetic Acid in 100% filtered Di-water; isocratic flow rate of U.5ml/min. Column Temp is 50C, RI temp is 35C. Injection volume is 10 μl.

Identification of genomic mutations Bacterial cells were grown overnight at 37° C. in veal infusion-yeast liquid medium for genomic DNA preparation by MasterPure Complete DNA purification kit. Sequencing was performed on Illumina Mi-Seq technology platform. Genome assembly was made using MIRA platform. Identification of single base mutation was made by comparison to parental strain genomic sequence.

### Example 2

### Sequences

Reference is made to the following sequences:

Genome: the genome of wild type BMS5 is SEQ.ID.NO.: 1. Due to its length, it is not reproduced in print.

alsS (5.1)
SEQ. ID. NO.: 2
vlakatnelktsgknrgaelwdclveqgvthvfgipgakidavfdalkdkgpelvIcrheqnaafmaaavgrltgkpgvclvtsg
pgasnlatglltantegdpwalagnviradrlkrthqsldnaalfqpitkysvevqetgnipeavtnafKaasaggagaafvsfp
qdwneitnykrwrsvpapkqgpapeeaysaaiakiqtaktpvtivgmkggrpeavkqirkllaktklpfvetyqgagtlsreled
qyfgriglfrnqpgdllleqadwltigydpieydpkfwnvngdraiihldeiqadidhayqpelellgdiaatvkqiehdavtfd
mgsreqevlselkqmltdsekapsdhksdrvhplqivqelrnaidddvtvtcdigshaiwmsryfrayeplkllisngmqtlgva
lpwaiaativnpgekvvsysgdggflfsameletavrtkapivhlvwndstydmvafqqmkkynrtscvdfgnidivkyaesfga
tglrvespeqtadvlqkglntegpviidipvdysdnvhIssdmtpkqfkekmkakal*
alsS (5.2) from B. licheniformis
SEQ. ID. NO.: 3
vlakatnelktsgknrgaelwdclveqgvthvfgipgakidavfdalkdkgpelvIcrheqnaafmaaavgrltgkpgvclvtsg
pgasnlatglltantegdpwalagnviradrIkrthqsldnaalfqpitkysvevqetgnipeavtnafraasaggagaafvsfp
qdwneitnykrwrsvpapkqgpapeeaysaaiakiqtaktpvtivgmkggrpeavkqirkllaktklpfvetyqgagtlsreled
qyfgriglfrnqpgdllleqadvvItigydpieydpkfwnvngdraiihldeiqadidhayqpelellgdiaatvkqiehdavtf
dmgsreqevlselkqmltdsekapsdhksdrvhplqivqelrnaidddvtvtcdigshaiwmsryfrayeplkllisngmqtlgv
alpwaiaativnpgekvvFvsgdggflfsameletavrtkapivhlvwndstydmvafqqmkkynrtscvdfgnidivkyaesfg
atglrvespeqtadvlqkglntegpviidipvdysdnvhIssdmtpkqfkekmkakal*
alsS (BMS5) (wildtype)
SEQ. ID. NO.: 4
vlakatnelktsgknrgaelwdclveqgvthvfgipgakidavfdalkdkgpelvlcrheqnaafmaaavgrltgkpgvclvtsg
pgasnlatglltantegdpwalagnviradrlkrthqsldnaalfqpitkysvevqetgnipeavtnafraasaggagaafvsfp
qdvvneitnvknvrsvpapkqgpapeeavsaaiakiqtakipvllvgmkggrpeavkqirkllaktklpfvetyqgagtlsrele
dqyfgriglfrnqpgdllleqadvvltigydpieydpkfwnvngdraiihldeiqadidhayqpelellgdiaatvkqiehdavt
fdmgsreqevlselkqmltdsekapsdhksdrvhplqivqelrnaidddvtvtcdigshaiwmsryfrayeplkllisngmqtlg
valpwaiaatlvnpgekvvsysgdggflfsameletavrtkapivhlvwndstydmvafqqmkkynrtscvdfgnidivkyaesf
gatglrvespeqtadvlqkglntegpviidipvdysdnvhlssdmtpkqfkekmkakal*
pta (5.1)
SEQ. ID. NO.: 5
vadlftkvqekvagkdvkivfpeglderilvavnnlagnkvlkpivvgnkediqakakelnitldgvdifdphtyegmeelvqaf
verrkgkateeqarkalldenyfgtmlvykgladglvsgaahstadtvrpalqiiktkegykktsgvfimargdeqyvfadcain
iapdsqdlaeiaiesantaqmfdidSrvamlsfstkgsaksdetdkvaeavkiakekapeltldgefqfdaafvpsvaekkapds
dikgdanvfvfpsleagnigykiaqrlggfeavgpilqglnmpvndlsrgcnaedvyntalitaaqal*
pta (BMS5) (wildtype)
SEQ. ID. NO.: 6
vadlftkvqekvagkdvkivfpeglderilvavnnlagnkvIkpivvgnkediqakakelnitldgvdifdphtyegmeelvqaf
verrkgkateeqarkalldenyfgtmlvykgladglvsgaahstadtvrpalqiiktkegykktsgvfimargdeqyvfadcain
iapdsqdlaeiaiesantaqmfdidprvamlsfstkgsaksdetdkvaeavkiakekapettldgefqfdaafvpsvaekkapds
dikgdanvfvfpsleagnigykiaqrlggfeavgpilqglnmpvndlsrgcnaedvyntalitaaqal*
bdhA (5.1)
SEQ. ID. NO.: 7
mkaarwhnqkdirienidepkaepgkvkikvkwcgicgsdlheylggpifipvgkphpltnemapvtmghefsgewevgegvkny
svgdrvvvepifathghqRaynldeqmgflglagggggfseyvsvdeellfklpeelsyeqgalvepsavalyavrqsklkagdk
aavfgcgpigllviealkaagatdiyavelsperqekakelgaiiidpsktddvveeiakrtnggvdvsyevtgvpwlrqaiqst
niagetvivsiwekgaeihpndivikertvkgiigyrdifpwlalmkegyfsadklvtkkivlddlieegfgalikeknqvkilv
kpn*
bdhA (BMS5) (wildtype)
SEQ. ID. NO.: 8
mkaarwhnqkdirienidepkaepgkvkikvkwcgicgsdlheylggpifipvgkphpltnemapvtmghefsgevvevgegvkn
ysvgdrvvvepifathghqgaynldeqmgflglagggggfseyvsvdeellfklpeelsyeqgalvepsavalyavrqsklkagd
kaavfgcgpigllviealkaagatdiyavelsperqekakelgaiiidpsktddvveeiakrtnggvdvsyevtgvpvvlrqaiq
stniagetvivsiwekgaeihpndivikertvkgiigyrdifpsvlalmkegyfsadklvtkkivlddlieegfgalikeknqvk
ilvkpn*
adhA (5.1)
SEQ. ID. NO.: 9
mcnnhptrvlsaphakakferttierralrphdilidikysgichsdihsafdewgggifpmvpgheiagvveavgeevttfavg
drvgvgcfvdscgeceyclngdeqyctkgvvqtynnldydgnptyggysqkivvtdrfvvripdqleldaaspllcagittyspl
khwnagpgkkvaivgmgglghlavqfahalgaevtvlsrsmnkkdealefgadhyfatsdpdtftelagrfdlitntvsantdvd
aylstlridgtlvnvgapakpdsysvfslitgrrsiaSslvggipqtqemldfaaehgiapkievipanqvdeayervlqsdvry
rfvidistl*
adhA (BMS5) (wildtype)
SEQ. ID. NO.: 10
mcnnhptrvlsaphakakferttierralrphdilidikysgichsdihsafdewgggifpmvpgheiagvveavgeevttfavg
drvgvgcfvdscgeceyclngdeqyctkgvvqtynnldydgnptyggysqkivvtdrfvvripdqleldaaspllcagittyspl
khwnagpgkkvaivgmgglghlavqfahalgaevtvlsrsmnkkdealefgadhyfatsdpdtftelagrfdlitntvsantdvd
aylstlridgtlvnvgapakpdsysvfslitgrrsiagstvggipqtqemldfaaehgiapkievipanqvdeayervlqsdvry
rfvidistl*
alsS (1.6)
SEQ. ID. NO.: 11
lnnvaaknetltvrgaelvvdsliqqgvthvfgipgakidavfdvlkdkgpelivcrheqnaafmaaavgrltgkpgvclvtsgp
gasnlatglvtantegdpwalagavkradrlkkthqsmdnaalf*
alsS (B. licheniformis BMS1) (wildtype)
SEQ. ID. NO.: 12
lnnvaaknetltvrgaelwdsliqqgvthvfgipgakidavfdvlkdkgpelivcrheqnaafmaaavgrltgkpgvclvtsgpg
asnlatglvtantegdpvvalagavkradrlkkthqsmdnaalfqPITKYSAEVEDANNIPEAVTNAFRAAASGQAGAAFLSFPQ
DVTAGPATAKPVKIMPAPKLGAASDEQISAAIAKIHNANLPVVLVGMKGGRPEAlEAVRRLLRKVKLPFVETYQAAGTLSHDLED
QYFGRIGLFRNQPGDMLLEKADVVLTVGYDPIEYDPVFWNGKGERSVIHLDEIQADIDHDYQPEIELIGDIAETLNHIEHDSLPV
SIDESFAPVLDYLKKALEEQSEPPKETKIDLVHPLQIVRDLRELLSDDITVTCDIGSHAIWMSRYFRTYRPHGLLISNGMQTLGV
ALPWAIAATLVNPGQKVVSVSGDGGFLFSAMELETAVRLKAPIVHIVWNDSTYDMVAFQQEMKYKRTSGVDFGGIDIVKYAESFG
AKGLRVNSPDELAEVLKAGLDAEGPVVIDIPVDYSDNIHLADQRFPKKFEEHFNKEASKQS*
dhbA (1.6)
SEQ. ID. NO.: 13
vkgkvalvtgasqgigkevalalagrgvfvaaadqnrkglleledeleqkglqgsgfaadMgdsaavdqliadiereigpidmlv
nvagvlrtglihstsdedwektfnvnstgvfnvsravarrmvprrtgaivtvgsnaaavprmhmaayaaskaaalmftkclglel
aeynircniispgstdtpmqrslwqceeaaqgviegsletfktgiplgklaspadiadavvfllsdgarhitmhdlrvdggatlg
a*
dhbA (B.licheniformis BMS1) (wildtype)
SEQ. ID. NO.: 14
vkgkvalvtgasqgigkevalalagrgvfvaaadqnrkglleledeleqkglqgsgfaadvgdsaavdqliadiereigpidmlv
nvagvlrtglihstsdedwektfnvnstgvfnvsravarrmvprrtgaivtvgsnaaavprmhmaayaaskaaalmftkclglel
aeynircniispgstdtpmqrslwqceeaaqgviegsletfktgiplgklaspadiadavvfllsdgarhitmhd
BMS7 16S sequence
SEQ. ID. NO.: 15
GTCAGTCAAACTACTTTATCGGAGAGTTTGATCCTGGCTCAGGACGAACGCTGGCGGCGTGCCTAATACATGCAAGTCGAGCGGA
CAGATGGGAGCTTGCTCCCTGATGTTAGCGGCGGACGGGTGAGTAACACGTGGGTAACCTGCCTGTAAGACTGGGATAACTCCGG
GAAACCGGGGCTAATACCGGATGGTTGTYTGAACCGCATGGTTCAGACATAAAAGGTGGCTTCGGCTACCACTTACAGATGGACC
CGCGGCGCATTAGCTAGTTGGTGAGGTAACGGCTCACCAAGGCGACGATGCGTAGCCGACCTGAGAGGGTGATCGGCCACACTGG
GACTGAGACACGGCCCAGACTCCTACGGGAGGCAGCAGTAGGGAATCTTCCGCAATGGACGAAAGTCTGACGGAGCAACGCCGCG
TGAGTGATGAAGGTTTTCGGATCGTAAAGCTCTGTTGTTAGGGAAGAACAAGTGCCGTTCAAATAGGGCGGCACCTTGACGGTAC
CTAACCAGAAAGCCACGGCTAACTACGTGCCAGCAGCCGCGGTAATACGKAGGTGGCAAGCGTTGTCCGGAATTATTGGGCGTAA
AGGGCTCGCAGGCGGTTTCTTAAGKCTGATGTGAAAGCCCCCGGCTCAACCGGGGAGGGTCATTGGAAACTGGGGAACTTGAGTG
CAGAAGAGGAGAGTGGAATTCCACGTGTAGCGGKGAAATGCGTAGAGATGTGGAGGAACACCAGTGGCGAAGGCGACTCTCTGGT
CTGTAACTGACGCTGAGGAGCGAAAGCGTGGGGAGCGAACAGGATTAGATACCCTGGTAGTCCACGCCGTAAACGATGAGTGCTA
AGTGTTAGGGGGTTTCCGCCCCTTAGTGCTGCAGCTAACGCATTAAGCACTCCGCCTGGGGAGTACGGTCGCAAGACTGAAACTC
AAAGGAATTGACGGGGGCCCGCMCAAGCGGTGGAGCATGTGGTTTAATTCGAAGCAACGCGAAGAACCTTACCAGGTCTTGACAT
CCTCTGACAATCCTAGAGATAGGACGTCCCCTTCGGGGGCAGAGTGACAGGTGGKGCATGGTTGTCGTCAGCTCGTGTCGTGAGA
TGTTGGGTTAAGTCCCGCAACGAGCGCAACCCTTGATCTTAGTTGCCAGCATTCAGTTGGGCACTCTAAGGTGACTGCCGGTGAC
AAACCGGAGGAAGGTGGGGATGACGTCAAATCATCATGCCCCTTATGACCTGGGCTACACACGTGCTACAATGGACAGAACAAAG
GGCAGCGAAACCGCGAGGTTAAGCCAATCCCACAAATCTGTTCTCAGTTCGGATCGCAGTCTGCAACTCGACTGCGTGAAGCTGG
AATCGCTAGTAATCGCGGATCAGCATGCCGCGGTGAATACGTTCCCGGGCCTTGTACACACCGCCCGTCACACCACGAGAGTTTG
TAACACCCGAAGTCGGTGAGGTAACCTTTTAGGAGCCAGCCGCCGAAGGTGGGACAGATGATTGGGGTGAAGTCGTAACAAGGTA
GCCGTATCGGAAGGKGCGGCTGGATCACCTCCTTTCTAAGGATTT

1. A Bacillus sp. strain which comprises at least one of the following characterizing sequences:
a) a 16S rDNA sequence with a sequence identity of at least 95%, and preferably, 100%, to the polynucleotide sequence according to SEQ ID NO:15, designated BMS7 16S;
b) a adhA sequence with a sequence identity of at least 95%, and preferably, 100%, to the polynucleotide sequence according to SEQ ID NO:9 designated adhA (5.1);
c) an bdhA sequence with a sequence identity of at least 95%, and preferably, 100%, to the polynucleotide sequence according to SEQ ID NO:7 designated bdhA (5.1);
d) a dhbA sequence with a sequence identity of at least 95%, and preferably, 100%, to the polynucleotide sequence according to SEQ ID NO:13 designated dhbA (1.6).
e) a pta sequence with a sequence identity of at least 95%, and preferably, 100%, to the polynucleotide sequence according to SEQ ID NO:5 designated pta (5.1).

2. The Bacillus strain according to claim 1 which is selected from the group of Bacillus subtilis and Bacillus licheniformis.

3. The Bacillus strain according to claim 1, wherein said strain is capable of overproducing acetic acid or acetate.

4. The Bacillus strain according to any of claim 1 selected from the group consisting of:
a) B. subtilis BMS 7;
b) a mutant of B. subtilis BMS 5, wherein said mutant comprises a DNA sequence identity to BMS 5 of at least 95%.

5. The Bacillus strain of any of claim 1 wherein said strain is capable of overproducing acetic acid or acetate by at least 40%, 50%, 60%, 70%, 80%, 90%, 100% compared to the wild type strain BMS 5.

6. The Bacillus strain of claim 1, wherein said strain inhibits the growth of C. prefringens bacteria.

7. A composition comprising the Bacillus strain or compounds obtained from the Bacillus strain of claim 1.

8. The composition of claim 7, wherein said composition is a feed-stuff and further comprises at least one further feed or food ingredient selected from the group consisting of: proteins, carbohydrates, fats, further probiotics, prebiotics, enzymes, vitamins, immune modulators, milk replacers, minerals, amino acids, coccidiostats, acid-based products, medicines, and combinations thereof.

9. A method of providing nutrients to an animal, inhibiting the growth of pathogenic bacteria or providing a healthier environment, comprising administering or applying the composition of claim 8.

10. The method of claim 9, wherein said method comprises feeding said composition to animals as a feedstuff in an amount sufficient to: improve the feed conversion rate of the animals; decrease the mortality rate of the animals; increase the survival rates of the animals; increase weight gain of the animals; increase the disease resistance of the animals;
increase the immune response of the animals; establish or maintain a healthy gut microflora in the animals; and/or reduce pathogen shedding through the feces of the animals.

11. A Bacillus strain according to claim 1, comprising an alsS sequence with a sequence identity of at least 95%, and preferably, 100%, to the polynucleotide sequence according to SEQ ID NO:2 designated alsS (5.1) or SEQ ID NO:3 designated alsS (5.2) and at least a second sequence selected from the group consisting of:
a) a 16S rDNA sequence with a sequence identity of at least 95%, and preferably, 100%, to the polynucleotide sequence according to SEQ ID NO:15, designated BMS7 16S;
b) a alsS sequence with a sequence identity of at least 95%, and preferably, 100%, to the polynucleotide sequence according to SEQ ID NO:2 designated alsS (5.1) or SEQ ID NO:3 designated alsS (5.2);
c) a adhA sequence with a sequence identity of at least 95%, and preferably, 100%, to the polynucleotide sequence according to SEQ ID NO:9 designated adhA (5.1);
d) an bdhA sequence with a sequence identity of at least 95%, and preferably, 100%, to the polynucleotide sequence according to SEQ ID NO:7 designated bdhA (5.1);
e) a dhbA sequence with a sequence identity of at least 95%, and preferably, 100%, to the polynucleotide sequence according to SEQ ID NO:13 designated dhbA (1.6).
f) a pta sequence with a sequence identity of at least 95%, and preferably, 100%, to the polynucleotide sequence according to SEQ ID NO:5 designated pta (5.1).

12. A Bacillus according to claim 11 comprising all the sequences a-f.
